(19) United States
(12) Patent Application Publication
(10) Pub. No.: US 2026/0337551 A1
(43) Pub. Date: Oct. 29, 2026

(54) CELL MACROENCAPSULATION DEVICES, METHOD OF FABRICATION AND USE THEREOF

(71) Applicant: THE ROYAL INSTITUTION FOR THE ADVANCEMENT OF LEARNING/MCGILL UNIVERSITY, Montréal (CA)

(72) Inventors: Corinne HOESLI, Québec (CA); Richard LEASK, Montréal (CA); Jonathan BRASSARD, Montreal (CA); Steven PARASKEVAS, Montreal (CA); Brenden MOEUN, Nepean (CA)

(21) Appl. No.: 19/113,510

(22) PCT Filed: Sep. 21, 2023
(86) PCT No.: PCT/CA2023/051249

§ 371 Date: Mar. 20, 2025

Related U.S. Application Data
(60) Provisional application No. 63/408,548, filed on Sep. 21, 2022.

(51) Int. Cl. C12N 5/00; A61K 9/00; A61K 35/39; A61M 27/00; C12N 5/071

(52) U.S. Cl. CPC C12N 5/0012; A61K 9/0024; A61K 35/39; C12N 5/0677; A61M 27/002; A61M 2207/00; C12N 2533/30; C12N 2533/74

(57) ABSTRACT

It is provided a cell macroencapsulation device for implanting cells in a recipient comprising cells encapsulated in a porous membrane, wherein the porous membrane consists of a sugar network coated with a polymeric solution comprising porogen, which can be used as a graft to transplant islets while protecting the graft from the immune system as well as providing enhanced convection and diffusion to ensure long-term cell survival and physiological insulin delivery.

### CROSS-REFERENCE TO RELATED APPLICATION

The present application is claiming priority from U.S. Provisional Application No. 63/408,548 filed Sep. 21, 2022, the content of which is hereby incorporated by reference in its entirety.

### TECHNICAL FIELD

It is provided a cell macroencapsulation device for implanting cells or cell aggregates in a recipient.

### BACKGROUND

Diabetes is a chronic disease characterized by the body's inability to regulate blood glucose. Insulin is a critical hormone for blood glucose regulation. Type 1 diabetes results mainly from an autoimmune attack and destruction of the insulin-producing beta cells found in pancreatic islets. Type 2 diabetes results mainly from deficient insulin sensing, but beta cell dysfunction also plays a critical role. In the last decades, diabetes has emerged as a silent pandemic, affecting more than 10% of adults in North America and 537 million people worldwide, a number expected to rise to 784 million within the next 25 years. The associated high blood glucose level significantly increases the risks for serious health problems such as cardiovascular diseases, neuropathy, and nephropathy, ultimately resulting in more than 6.7 million deaths annually and almost a trillion dollars in health expenditure in the US alone. Diabetes is currently treated using exogeneous insulin therapy, a market well over $30 billion annually with a compound annual growth rate above 4%. Insulin injections and artificial pancreases (insulin+glucagon pumps) fail to recreate physiological glycemic control and to eliminates diabetes complications, in addition to the burden on the patient associated with frequent blood glucose monitoring and care to avoid under or over-administration of insulin.

Islet transplantation has given new hope for a long-term treatment to type 1 diabetes, with over 70% of islet transplant recipients remaining insulin independent after 2 years. Islets of Langerhans or ‘islets’ are clusters of cells found in the pancreas which secrete insulin in response to different stimuli, including elevated blood glucose after meals. The secreted insulin which enters the blood stream then triggers glucose uptake and storage in other tissues and organs. Even when insulin independence is lost, the frequency of potentially life-threatening hypoglycemic events is greatly reduced in islet transplant recipients. More recently, stem cell derived beta cells have been delivered to patients during early clinical trials, showing promises in stem cell therapy for diabetes. These trials have shown that encapsulation devices, including those made from or containing alginate, are safe. To date, the only strategy that has shown efficacy of stem cell-derived pseudo-islets in humans was with non-encapsulated cells where immune suppression was required.

The risks associated with lifelong immune suppression needed to avoid graft rejection drastically reduce access to the therapy to only the most severe cases of type 1 diabetes (<1% of patients). Moreover, stem cell-derived grafts can contain unwanted cell types that may cause safety concerns if they are not contained in a potentially retrievable device. Overcoming these limitations would open the market for beta cell therapy to the 100 million people needing regular insulin injection worldwide, i.e. all type 1 diabetes and 10-25% of type 2 diabetes.

It is thus highly desired to be provided with new means for islet transplantation.

### SUMMARY

It is provided an encapsulation device fabrication methods to improve the survival and efficacy of therapeutic cells for diabetes.

These fabrication methods and resulting devices can also be used to treat other degenerative disease such as liver failure, kidney failure or thyroid defects.

These fabrication methods can also be used to culture encapsulated cells in 3D in vitro cultures.

In an embodiment, it is provided a method to manufacture a cell macroencapsulation device comprising the steps of preparing a porous membrane by coating a sacrificial template with a polymeric solution mixed with porogen; and encapsulating cells in the porous membrane.

It is also provided a cell macroencapsulation device comprising cells retained by a polymeric membrane fabricated through solvent casting a polymeric solution applied to a sacrificial template, with or without incorporation of porogens for particulate leaching.

In an embodiment, the sacrificial template is made of sugar.

In a further embodiment, the polymeric solution consists of a polycarbonate polyurethane dissolve into a volatile organic solvent.

In another embodiment, the sacrificial template is coated with 1 to 10 layers of the polymeric solution mixed with porogen.

In an embodiment, the sacrificial templates and the coatings are alternated to yield multiple compartments.

In a further embodiment, the porous membrane is created using a molded or a 3D printed template.

In another embodiment, the porous membrane is made in silicon elastomer.

In an embodiment, the porous membrane is molded as a pouch.

In a further embodiment, the pouch comprises a vasculature.

In another embodiment, the device consists of a carbohydrate glass template wherein a hot metal rod or needle is inserted therein to create a filling port after coating.

In an alternate embodiment, the porous membrane is further sterilized.

In an embodiment, the porous membrane is further dried before encapsulating the cells.

In a further embodiment, the cells are mixed with an immobilization material for encapsulation into the porous membrane.

In another embodiment, the porogen has particle sizes ranging from 1 microns to 1000 microns in size.

In an embodiment, the immobilization material is a hydrogel.

In an alternate embodiment, the hydrogel is immunoprotective.

In a further embodiment, the hydrogel has functional groups that allow cell adhesion, survival, promote differentiation, cell migration, angiogenesis or immunomodulation.

In an embodiment, the hydrogel is in the form of microbeads.

In an alternate embodiment, the immobilization material is a combination of more than one hydrogel.

In a further embodiment, the hydrogel is alginate, chemically-modified alginate, agarose, chitosan, collagen, Matrigel, fibrinogen, laminins, decellularized product, a polyethyelene-glycol based gel, or blends thereof.

In an embodiment, the cells are encapsulated using a membrane emulsification and internal gelation.

In an alternate embodiment, the cells are encapsulated using a nozzle-based encapsulation.

In a further embodiment, the cells are encapsulated using conformal coating.

In an embodiment, comprising a nanothin coating around the cells or cell aggregates.

In an embodiment, the cells are injected in the hydrogel through the membrane using a needle.

In an embodiment, the device described here produced by the method encompassed herein.

In an alternate embodiment, the pouch contains at least one subcompartment.

In a further embodiment, at least one subcompartment is a vascular graft or a channel.

In an embodiment, the polymeric solution used for solvent casting consists of a non-hydrolysable oil-soluble polymer dissolved into a volatile organic solvent.

In an alternate embodiment, the non-hydrolysable oil-soluble polymer is a polycarbonate or a polyurethane.

In a further embodiment, the non-hydrolysable oil-soluble polymer is polycarbonate urethane.

In an embodiment, the solvent is dimethylformamide or dimethyl acetamide.

In an alternate embodiment, the sacrificial template is made of sugar.

In a further embodiment, the sacrificial template is coated with 1 to 10 layers of the polymeric solution comprising porogen.

In an embodiment, the sacrificial template is coated with polycarbonate polyurethane mixed with dimethylformamide mixed with porogen.

In an alternate embodiment, the device encompassed herein further comprises an immobilization material.

In a further embodiment, the hydrogel is loaded with conjugated or adsorbed drugs, reporter molecules or contrast agents.

In an embodiment, the immobilization material is loaded with cells for generating blood vessels.

In an alternate embodiment, the immobilization material is loaded with angiogenic cells.

In a further embodiment, the immobilization material is loaded with endothelial progenitor cells, endothelial colony-forming cells, endothelial cells, mesenchymal stem/stromal cells, stromal cells, pericytes, microvessel fragments or combinations thereof.

In an embodiment, the angiogenic cells are autologous, allogeneic or xenogeneic

In an alternate embodiment, the device encompassed herein further comprises an oxygen source.

In a further embodiment, the oxygen source is an oxygen-rich gas or fluid, an oxygen-releasing material, or a detachable transcutaneous perfusion of oxygenated gas or liquid.

In an embodiment, the oxygen source is calcium oxide or peroxide.

In an alternate embodiment, the oxygen source incorporated into the immobilization material as microparticles.

In a further embodiment, the device described herein further comprises a detachable source of oxygen-carrying perfusate.

In an embodiment, the device described herein is further connected to a transcutaneous fluidic system.

In an alternate embodiment, the detachable transcutaneous perfusion is of pure oxygen, air, oxygenated saline solution, hemoglobin-loaded solution, heme-containing proteins, peptides, fluids or particles, or perfluorocarbons.

In a further embodiment, the detachable transcutaneous perfusion is for perfusing a solution that contain angiogenic growth factors.

In an embodiment, the device described herein further comprises an angiogenic material with or without angiogenic or immunomodulatory cells.

In an alternate embodiment, the cells implanted contain insulin-producing cells.

In a further embodiment, the insulin-producing cells are pancreatic islets.

In an embodiment, the cells are generated through differentiation or transdifferentiation.

In an alternate embodiment, the islets are from a cadaveric donor, stem cell-derived products or porcine islets.

In a further embodiment, the cells implanted are pancreatic beta cells, endothelial colony-forming cells, mesenchymal stem cells, stromal vascular fraction cells, megakaryocytes, pericytes, smooth muscle cells, hepatocytes, thyroid cells, hepatic cells, renal cells, fibroblasts, microvessel fragments or combinations thereof.

It is further provide the use of the device encompassed herein for treating type 1 diabetes treatment, liver disease, thyroid disease, or kidney disease.

In an embodiment, the device as described herein is used for an implantation as an as arteriovenous shunt or an interposition graft.

### BRIEF DESCRIPTION OF THE DRAWINGS

Reference will now be made to the accompanying drawings.

FIG. 1 illustrates general examples of islet macroencapsulation device designs as encompassed herein. All designs allow vascularization while maintaining graft immunoprotection. FIG. 1A shows a pouch made from a porous material filled with therapeutic cells that allows vascular ingrowth while retaining a protective barrier around the therapeutic cells. In the period prior to vascular ingrowth, a temporary oxygenation source can be provided through oxygen-releasing beads as shown in FIG. A1i or through oxygenated gas/liquid flow lines as shown in FIG. A1ii. FIG. 1B illustrates a device with active blood flow where oxygen and nutrients can be supplied immediately to the therapeutic cells by connection to the recipient vasculature. The external material can either be porous to allow vascular ingrowth from the host as shown in FIG. 1Bi or have low porosity to contain cells including red blood cells within as shown in FIG. 1Bii. In both cases (FIG. 1A and FIG. 1B) the therapeutic cells may be previously nanoencapsulated or microencapsulated to increase the pore size range of the pouch or device. Acronyms: ECFC—endothelial colony-forming cells; MSCs—mesenchymal stem/stromal cells.

FIG. 2 illustrates the fabrication process of pouch-based hybrid devices which combine microencapsulation and macroencapsulation. FIG. 2A shows pouch templates created through casting of a sacrificial material in a mold. Shown is carbohydrate glass cast into a polydimethylsiloxane mold generated through 3D printing. FIG. 2B shows carbohydrate glass templates which were de-molded, followed by inserting a hot needle and mounting on syringes. FIGS. 2C and D show pouches of different scales obtained after sacrificial material dissolution and needle removal. FIG. 2E shows a sectioned pouch after microencapsulated cell filling. FIGS. 2F and G show high viability of the encapsulated cells and immobilization of the microbeads in the angiogenic matrix.

FIG. 3 illustrates the device manufacturing, showing in FIG. 3A steps necessary for the fabrication of the vascular compartment. In FIG. 3B, steps for the fabrication of sugar molds for iterative molding and coating. In FIG. 3C, steps for the fabrication of the external pouch of the device.

FIG. 4 illustrates a method of fabrication of tubes or networks that can serve as internal device components. FIG. 4A is an illustration of the dip coating strategy with poly(carbonate urethane) and NaCl porogen. FIG. 4B are representative images of the grafts after dissolution of the sugar network and NaCl crystals. FIG. 4C are representative images of 4 and 9 branches channels with their corresponding cross-section to show hollow channels that can be perfused. FIG. 4D are representative scanning electron microcopy images of the cross-section and internal surface of the dip coated prosthesis. Scale bars, 500 μm. FIG. 4E is a graph showing the increase of longitudinal tensile strength in function of the number of layers. Mean and standard deviation of four different prostheses are shown. ****p<0.0001 and *p<0.05. FIG. 4F shows that the increase of internal radius of graft depends on the extent of internal pressurization. The mean and standard deviation of four different prostheses are shown. FIG. 4G is a stress-strain curve obtained using a biaxial tensile tester, showing 3 successive extension and release for 4 different prostheses. Circumferential and longitudinal axes are compared. FIG. 4H is a graph showing Young's Modulus in function of the axis (longitudinal vs circumferential) and the method of sterilization (ethanol or autoclaving). The mean and standard deviation of four different prostheses are shown. FIG. 4I is a graph of lactate dehydrogenase absorbance to assess the number of platelets adhering to the prosthesis during static incubation. The mean and standard deviation of 2 different prostheses for 2 different donors are shown. FIG. 4J shows electron micrographs of platelets adhered to a poly(carbonate urethane) prosthesis made by dip coating compared to a commercially available Impra® prosthesis.

FIG. 5 illustrates the effect of porogen particle size and concentration on polymer (poly(carbonate urethane)) coating properties. Factorial design of experiments was used to assess the effect of porogen particle size and concentration on the amount of polymer deposited during dip-coating (FIG. 5A) on expansion of the material after washes in water (FIG. 5B), on resulting porous polymer tube porosity (FIG. 5C) and longitudinal tensile strength (FIG. 5D). Bars represent the mean of 5 porous polymer tubes and standard deviation is shown for each. FIG. 5E are electron microscopy images of the cross-section of the various porous tubes obtained after dip-coating and dissolution of the carbohydrate glass and porogen in water as a function particle size and concentration of the salt. Scale bars, 100 μm.

FIG. 6 illustrates the intervascular device characterization, showing photographs of single channel (FIG. 6A) and 9-channel (FIG. 6B) devices and their inlets. In FIG. 6C, a graph is depicted showing Force-strain curves comparing the prosthesis alone vs the device. Standard deviation of 3 independent replicates is shown in lighter color. FIG. 6D, graph comparing the suture retention strength of chronoflex prosthesis and device to commercial alternative. Mean and standard deviation of five different samples are shown. FIG. 6E, graph showing the burst pressure of the prosthesis alone or the device with and without alginate filling. Mean and standard deviation of three or four different samples are shown for each condition. FIGS. 6F and G, fluorescent images taken using a small animal imager showing the diffusion of fluorescently labelled 20 kDa dextran through the prosthesis (FIG. 6F) and device (FIG. 6G). FIG. 7H, bioreactor set-up for perfusion of the cell therapy device inside a custom-made bioreactor. FIG. 6I, retrieval of the device after 48 hours of perfusion showing unobstructed lumen with stable alginate hydrogel inside the external compartment. FIG. 6J, GSIS for MIN6 inside the device with externally gelled alginate.

FIG. 7 illustrates material interaction with physiological components showing in FIG. 7A graph of LDH absorbance ratio to assess the number of platelets adhering to the prosthesis during static incubation. Mean and standard deviation of 4 different prosthesis for 4 different donors are shown. In FIG. 7B, graph of absorbance for WST-8 assay to assess the number of HUVECs adhering to the prosthesis and proliferating during static incubation. Mean and standard deviation of 4 independent biological replicates. In FIG. 7C, fluorescent images of confluent HUVECs on the internal surface of the prosthesis. Cells are labelled with DAPI and VE-cadherin. In FIG. 7D, photograph of a device perfused with human blood. In FIG. 7E, a photograph of the internal surface of the vascular prosthesis inside the device after 48 h of perfusion. In FIG. 7F, SEM image of the surface and cross-section after 48 h blood perfusion. In FIGS. 7G and H, hematoxylin and Eosin (FIG. 7G) or Sirius red (FIG. 7H) staining of tissue retrieve from mice after 28 days. In FIG. 7I, quantification of the width of fibrosis at different timepoint based on the porosity of the external surface. In FIG. 7J, sirius red staining of the graft after 28 days showing vascular channels with blood cells within (black arrows).

FIG. 8 illustrates maturation of stem cell derived islets inside the device. In FIG. 8A, brightfield images showing pseudoislets at day 10 of stage 7 either in suspension, in a static device or in a perfused device. FIG. 8B, representative live/dead staining of the pseudoislets after perfusion with calcein AM and Ethidium homodimer. In FIG. 8C, dithizone staining of the pseudoislets after 10 days of perfusion. In FIG. 8D, hematoxylin and Eosin staining of the pseudoislets after 10 days of perfusion. In FIG. 8E, flow cytometry results showing the percentage of the population expressing key pancreatic markers. Mean and standard deviation are shown for 3 independent devices with three independent differentiations. In FIG. 8F, fluorescent images of pseudoislets after perfusion. Cells are labelled with Nkx6.1 or Pdx1 and insulin or glucagon.

FIG. 9 illustrates device optimization and modeling, showing in FIG. 9A, COMSOL model showing cross-section of the device and oxygen concentration depending on cell density. In FIG. 9B, COMSOL model showing the profile of oxygen concentration inside the device through the different layers: Vascular channel, Polyurethane wall and alginate. In FIG. 9C, length of MIN6 survival from the internal (flow) versus external (static) side of the device. In FIG. 9D, representative live/dead staining of the cells with calcein AM and Ethidium homodimer. FIG. 9E, COMSOL model showing the effect of flow rate on the oxygen concentration at the outlet of the device. In FIG. 9F, experimental data showing the effect of flow rate on the oxygen concentration at the outlet of the device. Perfusion of a device with 40 millions cells/ml.

FIG. 10 illustrates transplantation into hybrid pigs of the device described herein, showing in FIG. 10A, incision to transplant vascular pancreas as an iliac arteriovenous shunt. In FIG. 10B, suture and transplantation of the device the day of the surgery. In FIG. 10C, device after 1 week in vivo. FIG. 10D shows an adapted curved device design to facilitate placement as iliac arteriovenous shunt.

FIG. 11 illustrates pouch-based hybrid device vascularization after two weeks of transplantation in C57BL/6 mice. Beta TC-tet cell aggregates were encapsulated in 5% alginate beads using a published stirred emulsification and internal gelation process. The beads were then mixed with a cold collagen solution and the mixture was injected into pouches with different porosity. The collagen was allowed to solidify prior to transplantation. The different porosities were created by introducing different size porogens into the polycarbonate polyurethane suspension during dip-coating of the pouch mold. Two weeks after transplantation subcutaneously, the pouches were removed from animals and processed for histology. FIG. 11a shows representative hematoxylin and eosin stained pouches at low magnification, indicating that pouches with porogen (NaCl) particles of 45-75 μm diameter display highest cellular infiltration into the pouch, with some cellular infiltration observed in smaller diameter porogen pouches but little to no infiltration for higher diameter porogen pouches. FIG. 11b, taken at higher magnification, shows that blood vessels (brown, identified through horseradish peroxidase-labelled anti-CD31 antibody) are formed within the pouch walls (dashed white lines). The highest frequency of blood vessels was found in the pouches created using 45-75 μm porogen particle diameters. FIG. 11c quantifies the number of nuclei observed in hematoxylin and eosin stained images, in the interior compartment of the pouches. Consistent with FIG. 11a, the highest cellular infiltration is observed with pouches fabricated using 45-75 μm porogen particle diameters.

### DETAILED DESCRIPTION

In accordance with the present disclosure, there is provided a macroencapsulation device for cell culture or transplantation created through solvent casting and particulate leaching applied to a sacrificial template.

It is described a novel cell encapsulation strategy to transplant islets while protecting the graft from the immune system as well as providing enhanced convection and diffusion to ensure long-term cell survival and physiological insulin delivery. The proposed cell delivery systems and device fabrication/assembly methods described herein are compatible with several islet sources including cadaveric donors, stem cell-derived products or porcine islets, as well as other cell types which secrete therapeutic molecules.

Accordingly, it is provided a method to manufacture cell macroencapsulation devices for in vivo implantation and delivery of a cell product. The manufacturing process, using successive steps of solvent coating and particulate leaching applied on successive sacrificial templates, allows modification of the device geometry and complexity to adapt to the delivery of specific cell products and implantation sites. As provided herein, it is encompassed multiple successive rounds of templating and coating to generate various compartments. Specific examples include a biomedical device containing multibranched vascular lattices for direct anastomosis and pouch-like devices with controlled geometry. The macroencapsulation device as encompassed herein can be impactful for the delivery of cell therapy products (such as islet transplantation in diabetes) or as an in vitro platform for long term culture of cells or cell aggregates such as organoids or pseudo-islets.

Accordingly, it is provided a vascularized cell encapsulation device for in vivo implantation or in vitro studies, method to fabricate it and the method to combine functional components within the device including but not restricted to (a) therapeutic cells with or without immunoisolation, (b) perfusable vascular networks, (c) oxygenation components and (d) angiogenic materials.

The encompassed device consists in a porous membrane with or without internal compartments generated through polymeric coating of a sacrificial template. The sacrificial template provides flexibility in device design and geometry, while the porosity of the polymeric material after particulate leaching facilitates nutrient, waste and secreted product diffusion. The final device can be used for in vitro testing and cultured or implanted. In one application, the device can be directly connected with the host blood circulation and injected with pancreatic cells. Pancreatic “pseudo-islets” can then regulate blood glucose levels by producing insulin in response to high glucose, providing an efficient treatment for diabetes. In other applications, cells in the transplanted graft can release other biologically active agents that will diffuse into the vascular prosthesis connected to the host blood stream to regulate physiological processes (hormones, small molecules or growth factors).

The pore size of the device can be tailored allowing, in some embodiments, vascularization through the pouch material. To achieve this, devices with porous membranes or membrane networks are fabricated by solvent casting and particulate leaching applied to a sacrificial template. The sacrificial template is first fabricated using 3D printing or molding. In an aspect, the sacrificial template is made from sugar and consists of one branch at one end that divides into 2 to 32 branches that converge back into one at the other end to create a membrane network. In other aspects, the geometry of the sacrificial template can be modified to generate vascular grafts or pouches with simple or complex geometries and subcompartments. The sacrificial template is coated with a polymer dissolved in a solvent in which the sacrificial template has low solubility. Porogen can be added into the polymeric solution before coating to obtain porous membranes, or omitted to obtain non-porous membranes. Drying allows removal of the solvent, leaving behind the coated sacrificial template. Dissolution of the sacrificial template and porogen particulates in a second solvent then yields a hollow porous polymeric network with one inlet that separates into several branches before converging into one outlet. In an aspect, the polymeric solution consists of a polycarbonate polyurethane dissolved into dimethylformamide or dimethyl acetamide. Porogen size and concentration can be controlled to fine tune pore size to tailor graft permeability to molecules or cells of different sizes. Non-porous membrane can also be created by omitting the porogen during the coating process. The thickness and mechanical properties of the graft can be controlled by the number of coating applied to the sugar network, varying from a thin coating with 1-3 layers to thick coating with >10 layers.

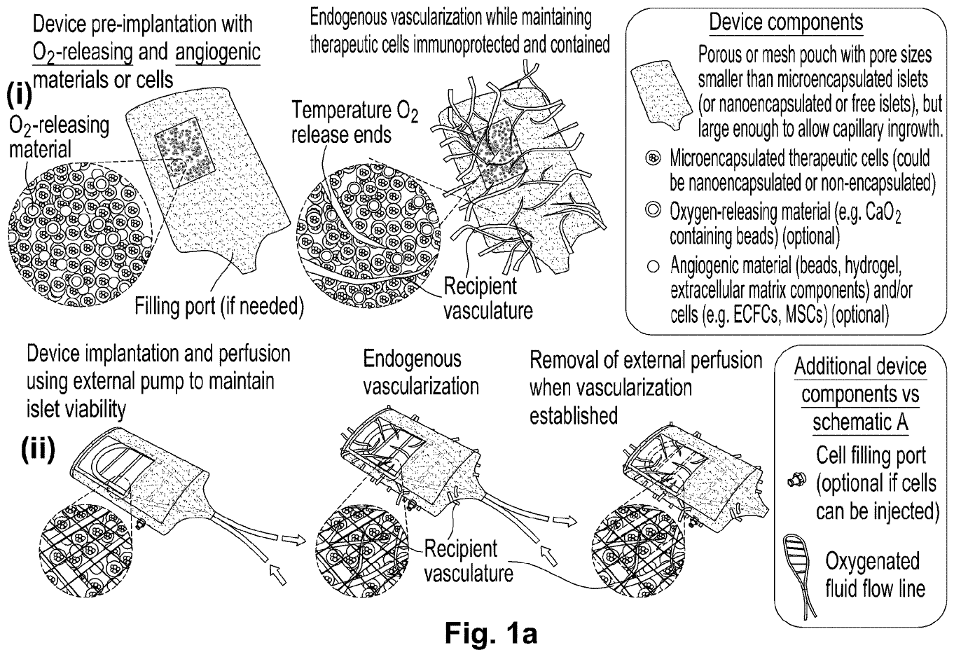

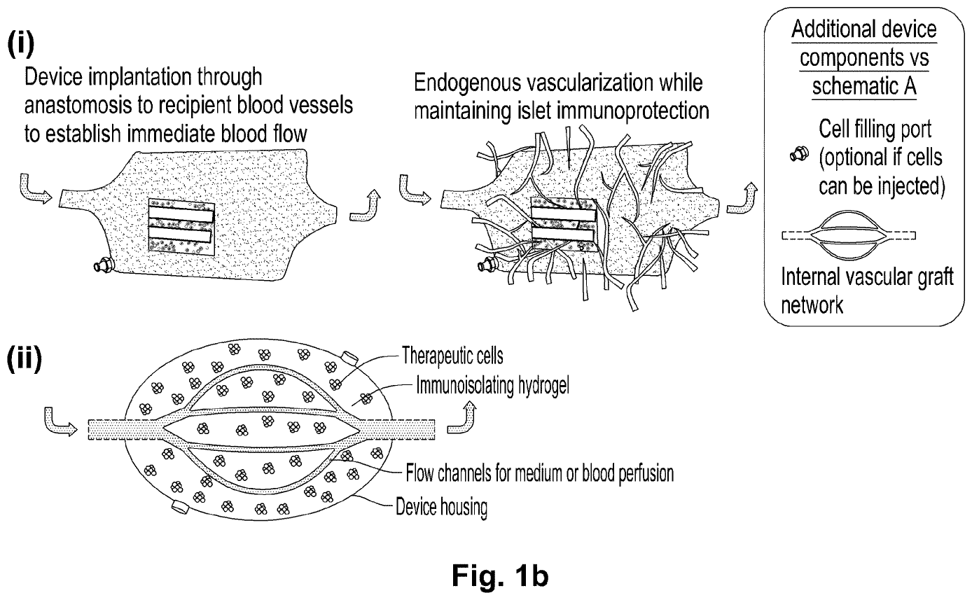

In one embodiment, as seen in FIG. 1A, the device consists in a porous pouch containing the cells to be transplanted. To protect the cells from the immune system and facilitate containment in the device, the therapeutic cells can previously be micro or nano-encapsulated, or be genetically engineered to reduce detection by the immune system. The pouch pore size allows vascular growth through the pouch material while entrapping the therapeutic cells within. By controlling the pore sizes of the external pouch, escape of higher-risk therapeutic cells such as those derived from pluripotent stem cells can be prevented. To achieve this, the solvent casting and particulate leaching methodology described above can be applied. A sacrificial, hemispherical sugar mold is dip-coated into a polymeric solution containing porogen particle sizes ranging from 1 microns to 100 microns in size. The pouch is then filled with a mixture of the immunoprotected therapeutic cells and an immobilization material such as extracellular matrix-derived proteins, biopolymers or synthetic angiogenic matrices that would facilitate vascular ingrowth and vascularization throughout the device. The immobilization material is then solidified or gelled and the pouch can be cultured in vitro or implanted. In some aspects, to improve vascularization, the immobilization material is loaded with cells that participate in generating new blood vessels such as endothelial progenitor cells and mesenchymal stem/stromal cells or other cell types that can act as pericytes. Notably, vascularization after implantation can take weeks and up to months to mature which leaves implanted cells without an effective mass transport system and source of oxygen. For devices where oxygen limitations occur prior to vascularization, this embodiment can also include a temporary oxygen source in the form of components that supply oxygen (FIG. 1Ai) or a detachable transcutaneous perfusion of oxygenated gas or liquid (FIG. 1Aii) to maintain the graft during vascularization. To accelerate ingrowth of blood vessels from the recipient, angiogenic materials with or without angiogenic or immunomodulatory cells can be added inside the pouch. In some embodiments, these angiogenic cells can be obtained from the recipient ahead of the device implantation procedure, for example by isolating ECFCs, MSCs or microvessel fragments from the recipient. The flow line can be transcutaneous and disconnected once the graft is vascularized and maintains function without external oxygenation. Examples of gases or liquids in this flow line can be pure oxygen, air, oxygenated saline solution, hemoglobin-loaded solution, or perfluorocarbons.

In another embodiment, as seen in FIG. 1B, the device consists in an internal vascular graft surrounded by an external pouch. A sacrificial template of the internal vascular graft is first obtained through methods such as casting or 3D printing, depending on the complexity of the desirable network.

Anastomosis to recipient blood vessels can be done through interposition, by-pass or shunting of recipient blood vessels. Microvasculature from the recipient can be established to improve graft function over time. Alternatively, an immunoprotective gel that does not allow microvessel ingrowth could be used if the internal vascular network is sufficient for graft survival and function. In an embodiment, the encapsulation device consists of the vascular graft inserted in a pouch sealed at the extremities of the vascular graft, creating a compartment around the vascular graft where cells and hydrogel can be injected. The external pouch is fabricated by encasing the previously described porous membranes or membrane networks inside a second layer of sacrificial material, based on the desired geometry for the final device. The composite is then coated again in the polymeric solution with or without porogen, depending on the desired mass transfer between the device and its environment. The sacrificial layer, often in the form of a capsule, is then dissolved, creating the space for cell and hydrogel encapsulation. In one aspect of the device (see FIG. 1Bi), the hydrogel can take different forms such as microbeads or nanocoatings and can be either immunoprotective or proangiogenic (enhancing vascularization and integration with the host). In another aspect of the device (FIG. 1Bii) the hydrogel used for the encapsulation of the cells serves as an immunoprotective barrier, preventing immune cell contact with the encapsulated cells while allowing nutrients and gas exchange.

The strategy presented successfully produces encapsulation devices with free control over the geometry of the device and internal compartments present in some embodiments. Accordingly, the device design encompassed herein is highly adaptable in geometry, permeability, and compartmentalization such that several components are modular within the device.

The fabrication method combining sacrificial materials with polymeric coatings to create intricate structures of controlled porosity embedded within a device using only one remaining material (i.e., both the device and embedded network can be made from the same material without suturing, gluing or fusing process needed).

The strategy proposed allows the development of a multibranched artificial organ that is scalable to human needs while avoiding cell hypoxia thanks to the parallelization of multiple channels inside the device and/or ingrowth of blood vessels from the graft recipient.

The strategy presented can be used with any sacrificial material that can either be 3D printed or molded and can be combined with a range of coating polymers and porogen particulates.

The strategy proposed is compatible with a wide range of active components, including cells of different origins and function, oxygen-releasing materials, and hydrogels for cell immobilization. The hydrogels can create a size exclusion barrier, or have functional attributes to support cell survival, function, differentiation, vascular ingrowth, or immunomodulation. Said hydrogels can be loaded or conjugated with drugs, reporter molecules or imaging contrast agents. Said hydrogel can either be injected and gelled (externally or internally) or it can be inserted as small beads that are individually protecting the cells. Two or more hydrogels can be included to impart combinations of features such as immunoprotection, immunomodulation, moieties to enhance islet survival or function or angiogenesis.

The device can be utilized for implantation or as an artificial organ for in vitro studies.

The devices can also be utilized without cells to model vascular systems or as branched vascular grafts.

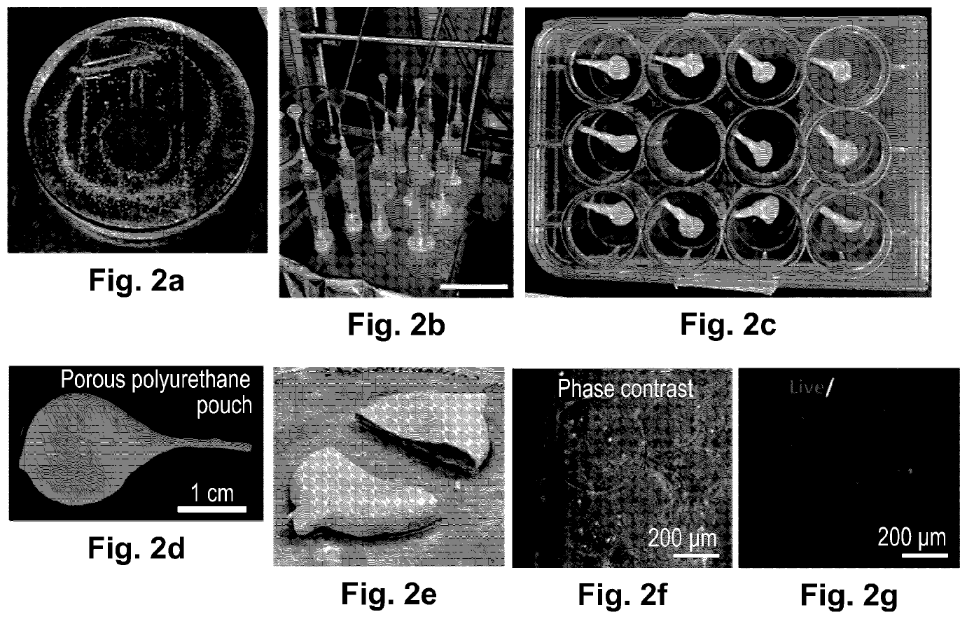

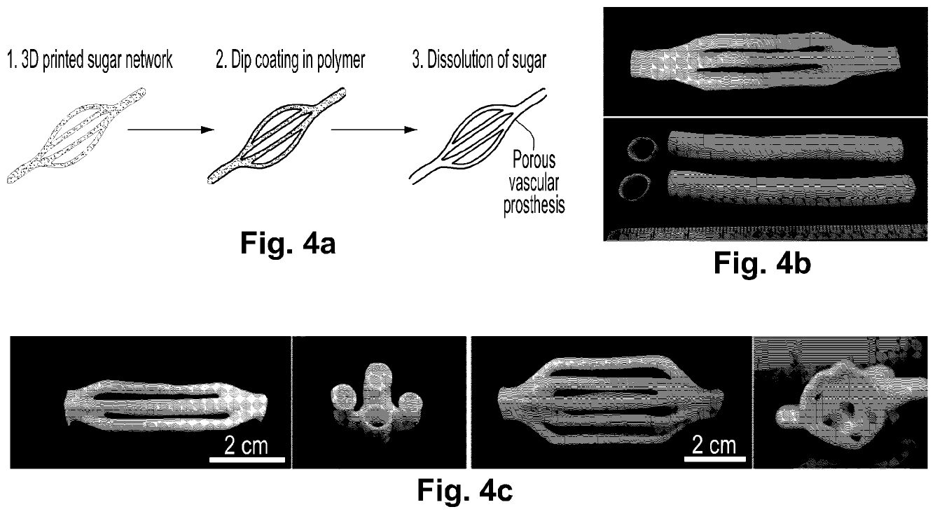

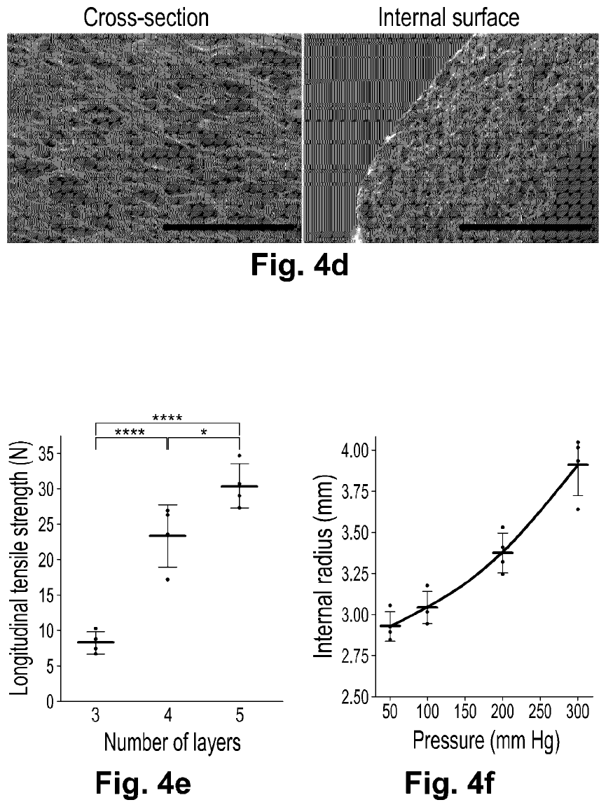

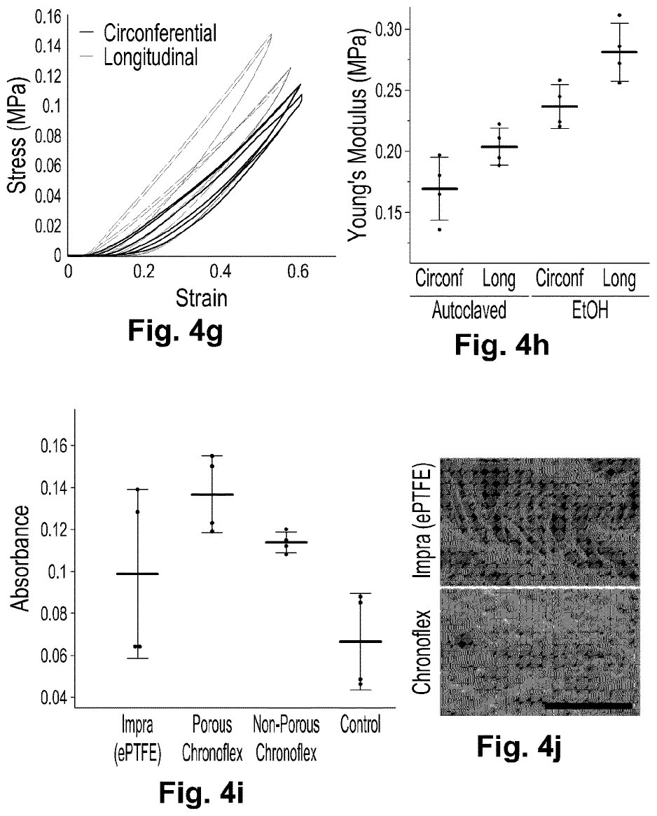

To obtain devices with relevant geometries, a sacrificial mold is first prepared using molding (FIG. 2) or 3D printing (FIG. 4) technologies. When simple sacrificial template geometries are needed, a casting method can be employed by: (1) fabrication of 3D-printed reverse molds, (2) reverse molding in silicon elastomer (polydimethylsiloxane—PDMS) and (3) casting of liquid sugar and subsequent solidification. Alternatively, complex carbohydrate networks can be obtained through a custom-made sugar 3D printer which act as templates for hierarchical vascular prostheses. Here, carbohydrate glass represent an ideal sacrificial material since it is nontoxic to cell and mammals, can be stored in laboratory conditions and can be coated at room temperature. Different saccharide compositions have been tested with varying concentration of dextrose and sucrose, with 100% sucrose creating a carbohydrate glass which is especially stable during storage. Other materials can also be used as sacrificial template, including polymers such as polyvinyl alcohol (PVA), thermoresponsive hydrogels such as gelatin or frozen liquids when used in a temperature-controlled environment.

As seen in FIG. 2B, shown are carbohydrate glass templates which were de-molded, followed by inserting a hot needle and mounting on syringes. The needles allow creation of a filling port compatible with later filling and closure of the pouches. The templates were then dip-coated in poly(carbonate-urethane) pre-mixed with porogen (NaCl microparticles of different sizes) at different concentrations to obtain different pore sizes. After 2 to 5 coating steps, the coated templates were left to dry overnight. The next day, the sacrificial material was dissolved through several washes in distilled water until the carbohydrate glass was completely dissolved.

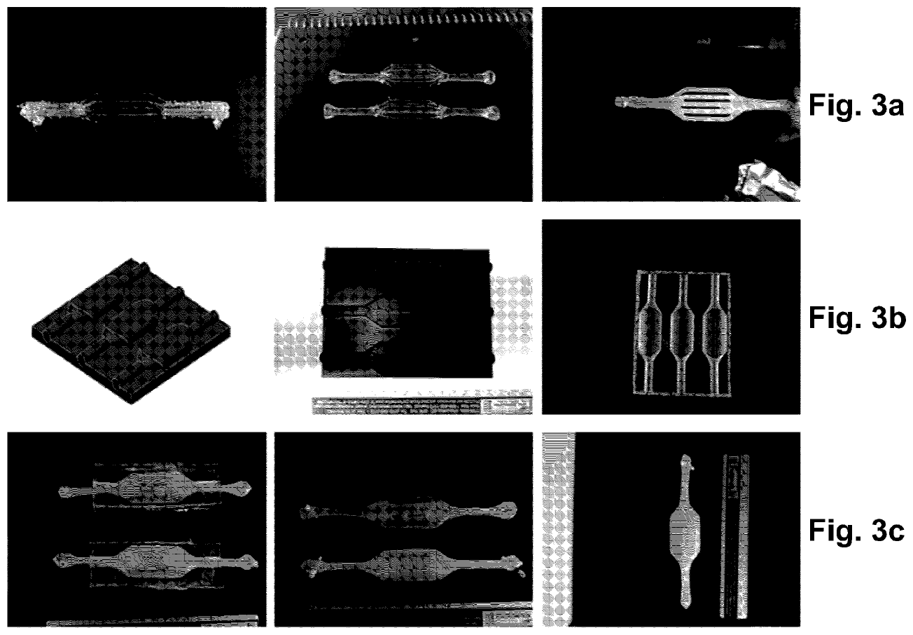

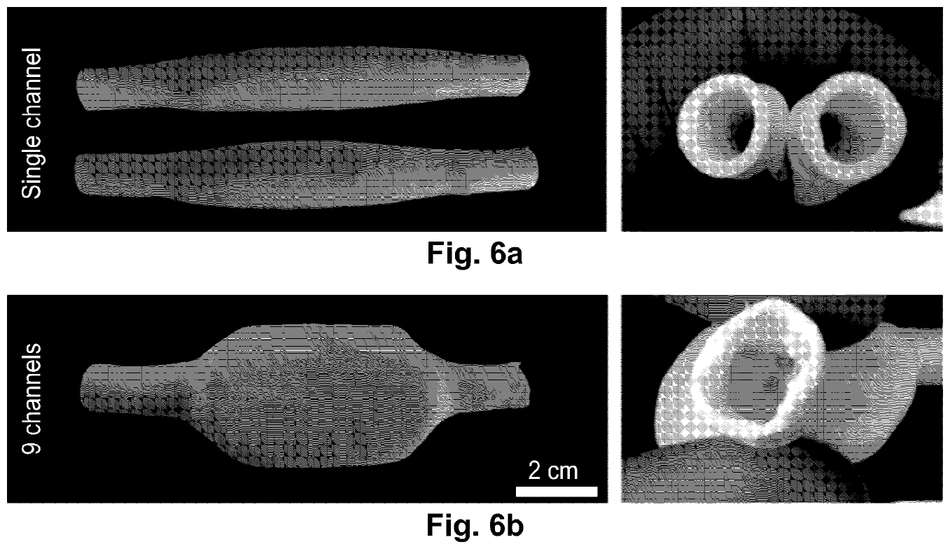

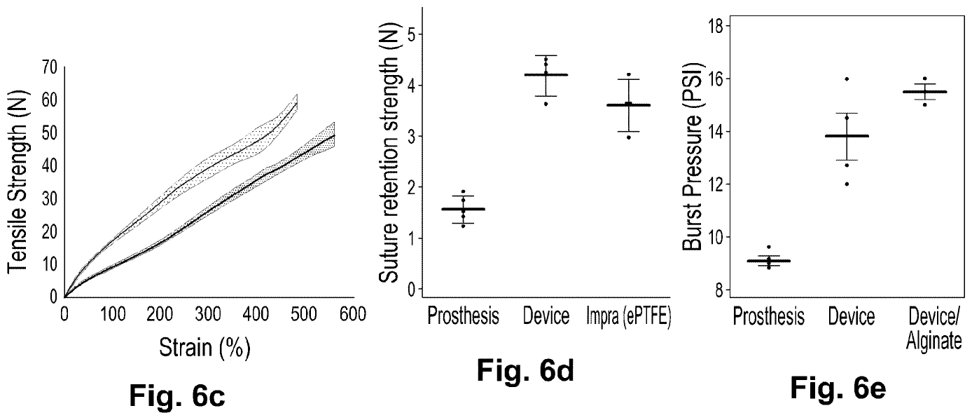

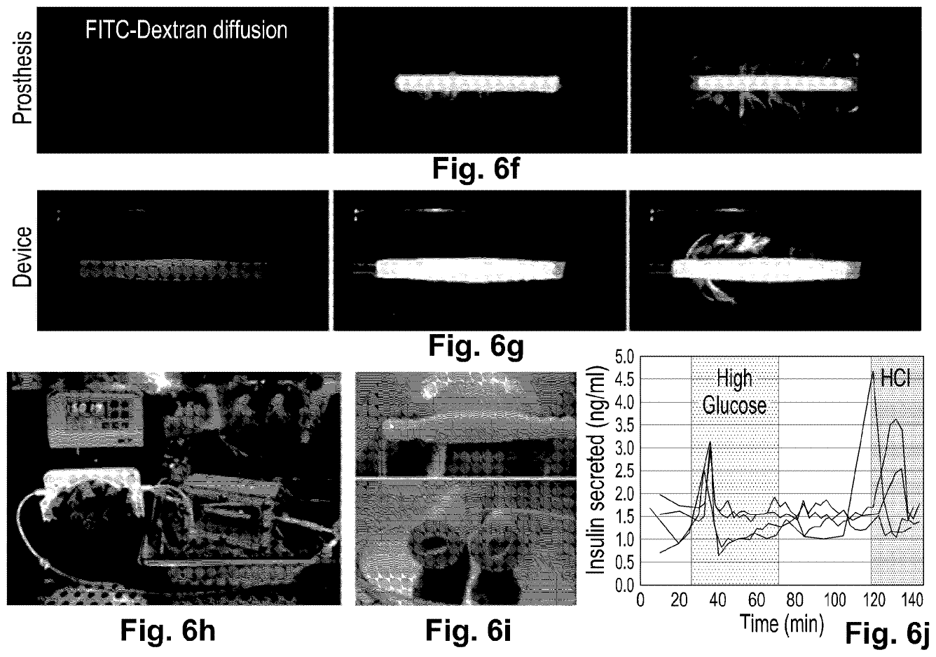

As depicted in FIG. 3, shown are carbohydrate glass templates that were first 3D printed and polished under flame, before being coated by a polycarbonate polyurethane solution containing porogen. PDMS molds for the fabrication of external components were fabricated by reverse molding 3D printed polylactic acid (PLA) constructs. Carbohydrate glass was then injected in the PDMS mold around the initial compartment (vascular in this case). After solidification, the new construct was demolded, coated again with polymer-porogen solution before being immersed in water to dissolve the sugar and porogen, resulting in final device with an internal compartment shown in FIG. 6B. A detailed description of the steps shown in FIG. 3 are as follows. Step 1: 3D printing of carbohydrate glass lattices (left panel of FIG. 3A) using a computer-assisted design model, polishing under a flame (middle panel of FIG. 3A) to remove small imperfections and create smooth surfaces without changing the main features, dip coating the printed templates with a well-mixed polycarbonate polyurethane and porogen suspension in solvent, drying to evaporate the solvent between each coating to obtain a polymer-coated vascular template (right-hand panel of FIG. 3A). Step 2: in parallel, a reverse mold is created through computer assisted design (left panel of FIG. 3B). The reverse mold is 3D printed using PLA (middle panel of FIG. 3B) or other thermoplastic or 3D printable materials that solidify at room temperature and allow printing of fine features. The reverse mold can also be obtained through machining or using an object such as tubing. The mold is then created by casting PDMS around the reverse mold (right-hand panel of FIG. 3B). Two molds can be assembled allowing placement of the vascular template between the two molds. These molds can be symmetric or have different features to create asymmetric outer device compartments. Another material than PDMS can be used, but it must be heat-resistant and have low water content if it is to be used with a heated hydroscopic material such as carbohydrate glass during the casting step. Step 3: The outer compartment is created by placing the vascular template within the mold or, in this case, between two external molds (left panel of FIG. 3C), casting carbohydrate glass into the mold filling the space that is left free by the vascular template (middle panel of FIG. 3C), demolding after cooling and solidification of the cast, and then dip-coating the completed structure in polycarbonate urethane which may or may not contain porogen (right-hand panel of FIG. 3C). After allowing evaporation of the dip coating solvent, the carbohydrate glass present in the vascular template and the outer compartment cast, as well as the porogen, are removed through dissolution in repeated water washes and incubations. This assembly is then autoclaved, filled aseptically with cell suspension in hydrogel and used for in vitro or in vivo studies.

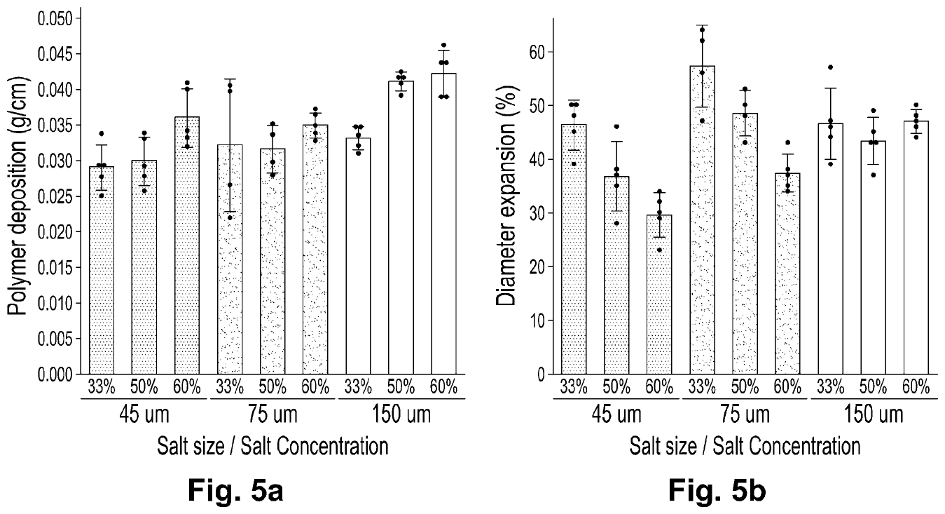

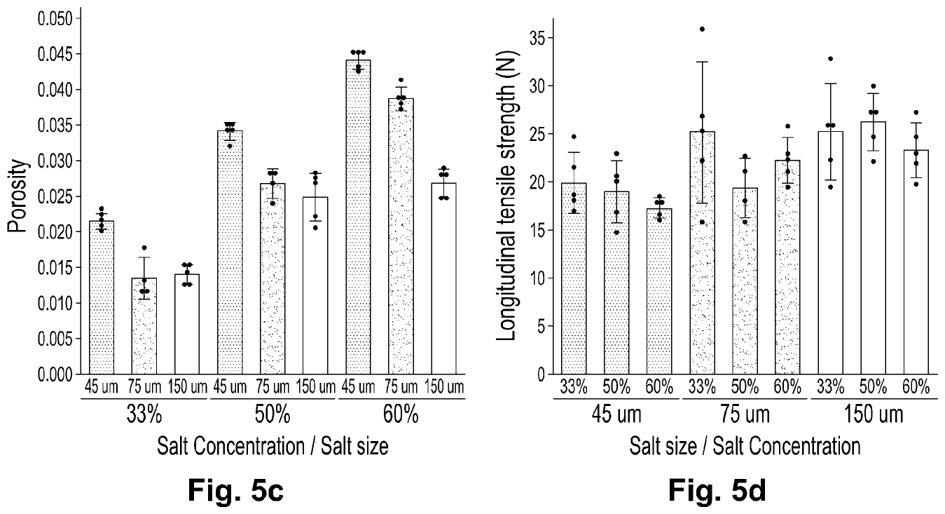

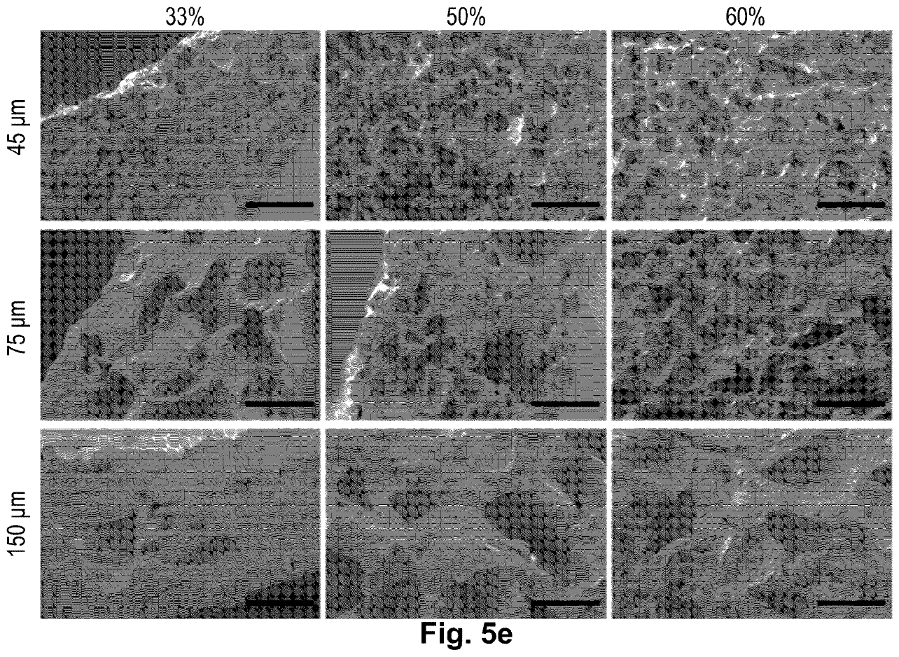

Using the dip coating of sacrificial sugar network approach (FIG. 4), a surface response design was created to simultaneously probe the effect of porogen particle size (three categories; S<45 μm, 45 μm<S<75 μm, 75 μm<S<150 μm) and concentration (NaCl:Polymer w/w ratio; C=33%, 50%, 60%) on the properties of a tubes generated through dip-coating, including reproducibility of the coating, porosity and swelling of the graft, as well as their tensile strength. Based on full factorial model with least squares fit, the thickness of a single coating increased when the concentration (p=0.0001) or size (p<0.0001) of the porogen was increased (FIG. 4E). The volumetric porosity of the porous tube increased with increasing salt concentration (p<0.0001) and decreasing salt size (p<0.0001; FIG. 5C). Surprisingly, the longitudinal tensile strength of the porous tube was not significantly affected by the porogen concentration (p=0.14; FIG. 5D), possibly because the strength at rupture was not corrected for the wall thickness.

To obtain a porous scaffold that will not hinder diffusion of glucose, insulin and other nutrients or secreted molecules through the prosthesis, a porogen is added to the solution prior to dip coating. Sodium chloride (NaCl) is an ideal porogen since it does not react with most of the dip coating solvents like dimethylformamide or dimethylacetamide. Other porogens can also be used, provided they do not dissolve in the solvent used for coating. After several sequential dip coatings of the sugar construct and evaporation of the solvent, both the sugar (sacrificial template) and the salt (porogen) can be washed away using multiple washes in distilled water, resulting in a porous device of defined geometry. The resulting porous devices ban be sterilized through various non-destructive methods. Autoclaving porous tubes generated through this method decreased the elastic modulus (FIG. 4H) but did not significantly affect the porosity. Devices can also be sterilized using ethanol or ultraviolet light, and we anticipate that other methods such as ethylene oxide or gamma irradiation would also be compatible with our fabrication method and materials.

Prior to use in cell studies, pouches were further dried at 50 °C for at least 30 min to remove traces of the solvent used during coating. Pancreatic beta cells (MIN6 cells) were microencapsulated using stirred emulsification and internal gelation, mixed with cold collagen I or Matrigel solution (the angioenic material), and infused into the pouch through the filling port (FIG. 2E). At this step, it would be possible to introduce other microbeads into the mixture such as oxygen-releasing microbeads. In some experiments, endothelial cells were introduced into the angiogenic material. The angiogenic material was then gelled by placing pouches at 37 °C inside a cell culture incubator for at least 30 min. Immediately after pouch filling and collagen/Matrigel setting, high viability of the encapsulated cells is observed, and the microbeads are immobilized in the angiogenic matrix (FIGS. 2F and G).

For devices where vascular ingrowth from the recipient is desirable, the porogen size should be selected such that capillaries can grow through the pouch material to allow nutrient, oxygen, waste and secreted product (e.g. insulin) transport. However, the porogen size should not be large enough to create pores through which the therapeutic cells can easily exit. Prior nanoencapsulation or microencapsulation of the therapeutic cells therefore increases the range of acceptable porogen sizes by allowing graft entrapment even at larger pore sizes. Mixing of the cells in higher-viscosity solutions such as alginate was also found to hinder cell escape from devices during loading and culture.

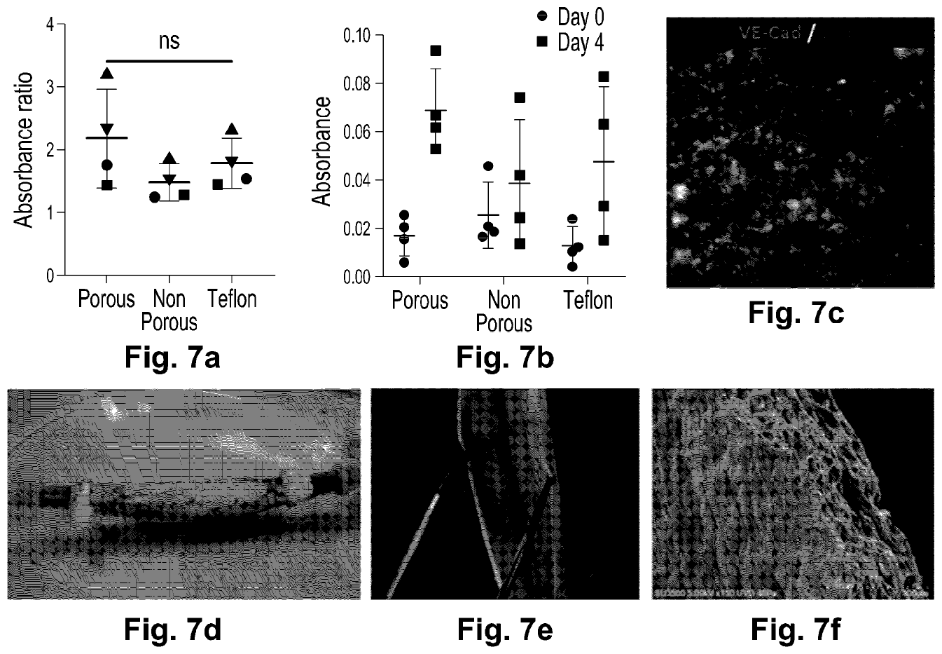

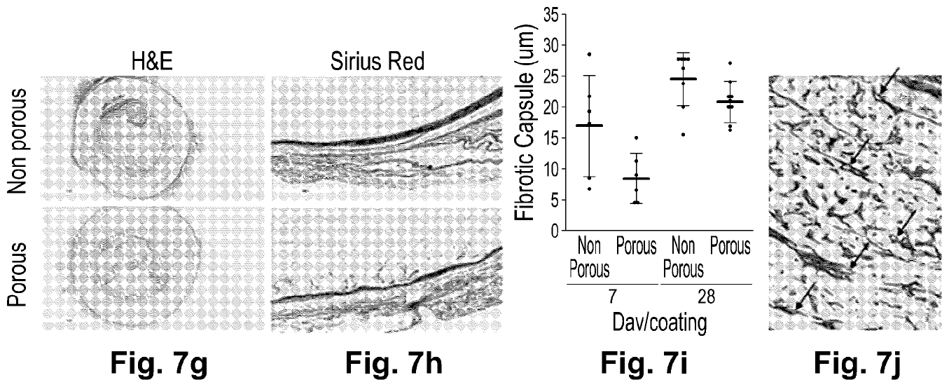

To evaluate fibrotic responses to device materials and the formation of vasculature within the device material, sections of tubular devices with different porosity (no porogen vs 45 μm diameter porogen) were autoclaved in water and then implanted into C57BL/6 mice. Histology section stained with hematoxylin and eosin or Sirius red showed little fibrosis around the implants, with the non-porous surface showing thicker fibrous encapsulation (p=0.019) than for porous grafts (FIGS. 7G and 7H). Implants retrieved after 28 days also showed cellular infiltration and some vascularization based on red blood cell staining within the device material pores (FIG. 7J). These results suggest that porous devices will trigger low levels of fibrosis after 4 weeks and allow vessel growth through device pores even when porogens below 45 μm are used.

As provided herewith, in one implementation, pouches were designed to allow infusion of pseudo-islets with or without prior nano or microencapsulation. Using the polymer-porogen dip-coating method, porous pouches with a cell seeding port were created by using carbohydrate glass hemispheres as templates (see FIG. 2). A cell seeding port was created either by incorporating this feature in the sacrificial mold, or by inserting an object such as a needle into the sacrificial material. After dip-coating in polymer-porogen mixtures, drying and washing to remove the sacrificial material and porogen, pouches of different sizes and shapes were obtained. By optimizing the porogen size and amount, the external pouch material can allow vascular ingrowth, while avoiding cell losses during filling. Prior nano or microencapsulation of the pseudo-islets can provide an immune barrier. This design allows blood vessels from the host to reach very short distances from graft cells, while still avoiding direct contact with immune cells present in blood. The external pouch also allows to contain and retrieve the graft, and mixing of other functional components with minimal changes to the device design or filling process. The cells, with or without prior microencapsulation or nanoencapsulation, can be mixed with extracellular matrix components, oxygen-releasing materials, angiogenic or drug-releasing materials, or imaging contrast agents in different forms such as gels, filaments or particles prior to device filling. In one embodiment, pouches have been assembled as follows: (1) encapsulation of beta cell aggregates, (2) mixing of the encapsulated cell aggregates within an immobilization material such as cold Matrigel or collagen, (3) injection of the mixture into the pouch through the seeding port (4) gelation of the immobilization material by placing the pouches into a cell culture incubator and (5) cell culture or transplantation. Cells survive the pouch assembly process (see FIG. 2G). Pouches can be transplanted into different sites such as the subcutaneous space, into the omentum, into the peritoneal cavity or other transplantation sites by methods known to those skilled the art.

The solvent casting and particulate leaching method applied to sacrificial templates can also be used to fabricate more complex compartmentalized devices or devices with complex geometries (see FIG. 4B). For devices that are to be connected to the recipient vasculature, the device material should be sufficiently porous to efficiently allow nutrient diffusion through the wall in contact with blood, while being strong enough to withstand blood flow and surgical procedures. Based on the results of the described surface response design (FIG. 5), salt particles below 45 μm and 60% weight ratio with the polymer as the porogen can maximize porosity while retaining adequate mechanical properties for applications as vascular implants (FIGS. 4E-I). Using these conditions, single tubular prostheses were fabricated as well as prostheses with 4, 6 and 9 branches that can be simultaneously irrigated by one entry and one exit point (see FIGS. 5B and C). Scanning electron microscopy confirmed high porosity at the surface of the graft as well as through the wall cross-section (FIG. 5D). The prostheses were characterized using the international standard ISO 7198 for Cardiovascular implants and extracorporeal systems—Vascular prostheses—Tubular vascular grafts and vascular patches. Three coating layers produced grafts that had defects leading to early rupture, whereas four or five coating produced strong prostheses with a longitudinal tensile strength above 20 N (FIG. 4E). The radial expansion of the graft under increasing pressure was then quantified (FIG. 4F). In the range most relevant to physiological blood forces (50-100 mm Hg), the grafts had a compliance of 7.47±0.32%/mm Hg, a result that is slightly higher than carotid and coronary arteries (~4-6 mm Hg). Using a biaxial tensile tester, the elasticity of the graft was compared in the longitudinal and circumferential direction. It was found that the longitudinal axis was consistently stronger than the radial axis, with a Young's Modulus of 0.281±0.024 MPa versus 0.242±0.017 MPa respectively (FIGS. 4G and H).

Using a similar approach with a combination of sacrificial sugar molding and dip coating, a cell encapsulation device was produced as described herein where a cell suspended in a hydrogel can be injected in a compartment surrounding the porous vascular prosthesis (FIG. 6A). To avoid a build-up of stress created by uneven deformation of the material, the pouch surrounding the prosthesis was created using the same combination of salt concentration and crystal size. The last coating step omitted porogen to obtain a blood-impermeable outermost coating and external compartment, while retaining the permeability of the internal vascular network. This resulted in a cell encapsulation device that can accommodate any volume from small (0.5-2 ml, FIG. 6A) to medium (5-10 mL, FIG. 6B) to large (up to 100 ml) of cell suspension either alone or embedded in hydrogel solution. The size of the external pouch can be tailored to the application and the geometry of the internal vascular prosthesis. In the case of the channel device, the longitudinal tensile strength and the burst pressure were increased (FIGS. 6C and D). Interestingly, the suture retention strength was also increased significantly, reaching similar values compared to commercially available grafts (FIG. 6E). Importantly, rapid diffusion of 20 kDa fluorescently labelled dextran was validated through the wall of the prosthesis (FIG. 6F) and through the complete device in the absence of cells or hydrogel (FIG. 6G).

Furthermore, the activation and adhesion of platelets on the prosthesis compared to commercially available expanded polytetrafluoroethylene (ePTFE) graft (Impra®) was also investigated. Based on lactate dehydrogenase release and SEM, no statistical difference in platelet adhesion on the provided material compared to ePTFE grafts made by dip coating of the sugar construct was observed (see FIGS. 4I and J).

As encompassed herein, the hydrogel can consist of various natural or synthetic polymers, with or without functional modifications known to those skilled in the art. As provided herewith, alginate is used as the encapsulation hydrogel. The low cost and ease of gelling in physiological conditions of the material presents advantages for human-scale devices. Aside from alginate, other examples previously used for islet delivery include chemically-modified alginate, agarose, chitosan, polyethyelene-glycol based gels, with or without addition of cell adhesion or immunomodulatory functional groups attached covalently or adsorbed to the gel, allowing controlled release in some cases, or blends of the aforementioned gels.

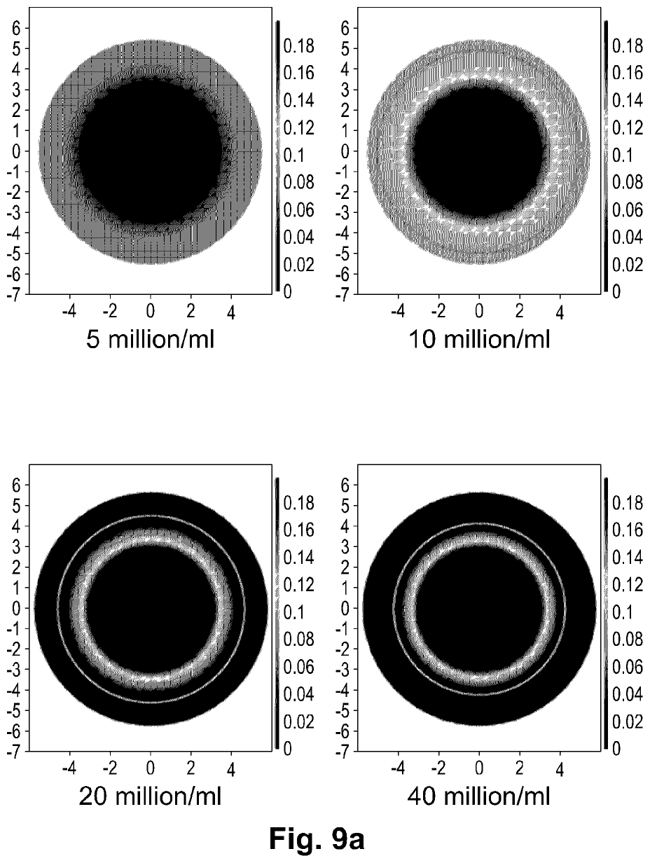

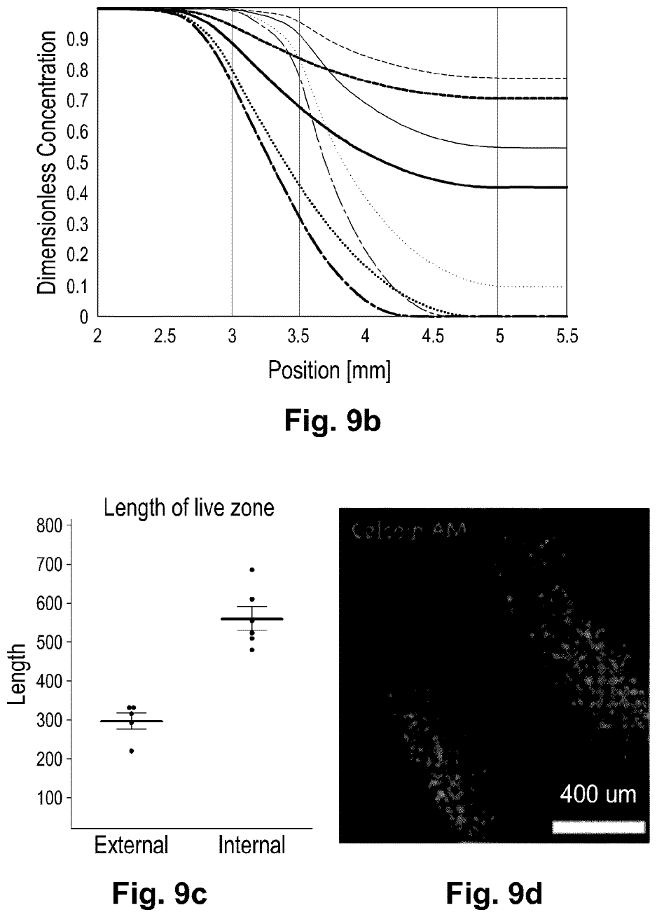

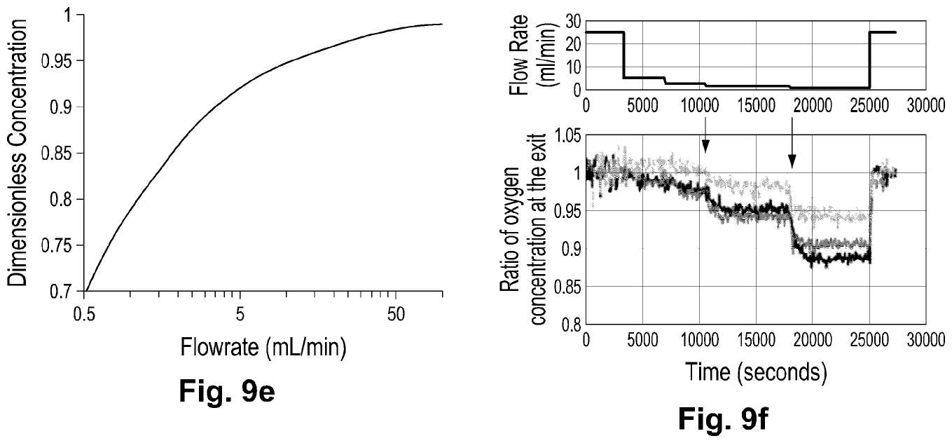

To validate survival of cells within the device, a mouse insulinoma cell line (MIN6) was used combined with internally or externally gelled alginate. Briefly, cells and hydrogel precursors can be directly injected inside the compartment surrounding the prosthesis (and gelled by supplying calcium ions to crosslink the alginate chains, either through dissociation of encapsulated calcium carbonate particles using an acidic solution (internal gelation) or by immersing the device into a calcium bath (external gelation). Gelation solution can also be circulated through inner vessels for more uniform gelation of larger devices. To mimic the islet morphology and enhance beta cell function, aggregates can be produced using a microwell system, resulting in robust production of similarly sized MIN6 pseudo-islets. Once cells have been encapsulated inside the device, it can be mounted within a custom-made bioreactor and perfused with culture medium, for several days and at flow rate reaching at least 500 mL/min (FIG. 6H). It is further provided that the device maintains an unobstructed lumen and stable alginate filling during perfusion for at least 48 hours (FIG. 6I). The device can then be recuperated, cut into sections and imaged for cell viability. Survival of MIN6 aggregates was observed to be comparable to the static controls. In addition, the provided device is compatible with external alginate gelation and, similarly, allows survival of MIN6 cells and perfusion with different amounts of glucose to trigger insulin secretion. Glucose stimulated insulin secretion (GSIS) assays performed under perfusion and showed rapid kinetics of insulin secretion both at high glucose concentration and with KCl addition (FIG. 6J). Oxygen models have also been developed to design the device and its internal vasculature to maximize cellular viability at higher concentration (FIG. 9). Flow rate, cellular density, diffusion coefficient and device geometry can all be modified to tailor device design to maximize survival and function. Human islets were also cultured in the device for 7 days to demonstrate viability of primary cells in the bioreactor and device.

Although the current designs were developed for islet and beta cells for type 1 diabetes treatment, this device can be loaded with a variety of other therapeutic cell types. Other therapeutic cells can include but are not limited to hepatocytes, thyroid cells, renal cells that could be used to treat liver, thyroid, and kidney disease. These are cells that can be implanted to deliver proteins that are missing in the body. Different cell types that are involved in generating blood vessels as a means of accelerating and improving the vascularization throughout these devices can be used alone or in combination with aforementioned therapeutic cells. Cell types that aid in vascularization can include, but are not limited to endothelial colony-forming cells, mesenchymal stem/stromal cells, stromal vascular fraction cells, pericytes, smooth muscle cells, and fibroblasts. Sertoli cells or mesenchymal stem/stromal cells can create an immunopriviledged environment to induce tolerogenic immune responses. The cells can be of autologous (from the recipient), allogeneic (from the same species) or xenogeneic (from another species) origin. The cells can be obtained directly from a donor, or can be obtained through differentiation or transdifferentiation of other cell types.

In addition to Matrigel and collagen, materials such as those containing laminins, fibrinogen, and decellularized products can be used to immobilize the implanted therapeutic cells and allow for endogenous vascularization.

Aside from perfusion with blood, other embodiments with oxygenation strategies that could be used to nourish the graft while vascularization occurs would include components that act as oxygen sources. These components include but are not limited to oxygen-rich gases or fluids, as well as oxygen-releasing materials. Examples of oxygen-rich gases or fluids are oxygen-rich gas, perfluorocarbons, or fluids which contain heme groups such as hemoglobin, leghemoglobin or other heme-containing proteins which can be included in a device subcompartment. Examples of oxygen-releasing materials are calcium oxide and various peroxides which can be incorporated into the immobilization material as microparticles. Another embodiment with another oxygenation strategy would be a detachable source of oxygen-carrying perfusate. Perfusable networks like those shown in FIG. 1 can be temporarily connected to a transcutaneous pumping system instead of the host circulatory system to facilitate other means of implantation (e.g., subcutaneously). These networks can be perfused with liquids other than blood to supplement the implant with oxygen and other advantageous molecules such as growth factors and proteins that can be used to facilitate stem cell differentiation, improve graft vascularization, and support cell function. These biomolecules can be dissolved in the perfusate along with soluble oxygen-carriers such as perfluorocarbons and other blood substitutes. Once the graft is adequately vascularized and/or the implanted cells are differentiated, the external perfusion system can be detached.

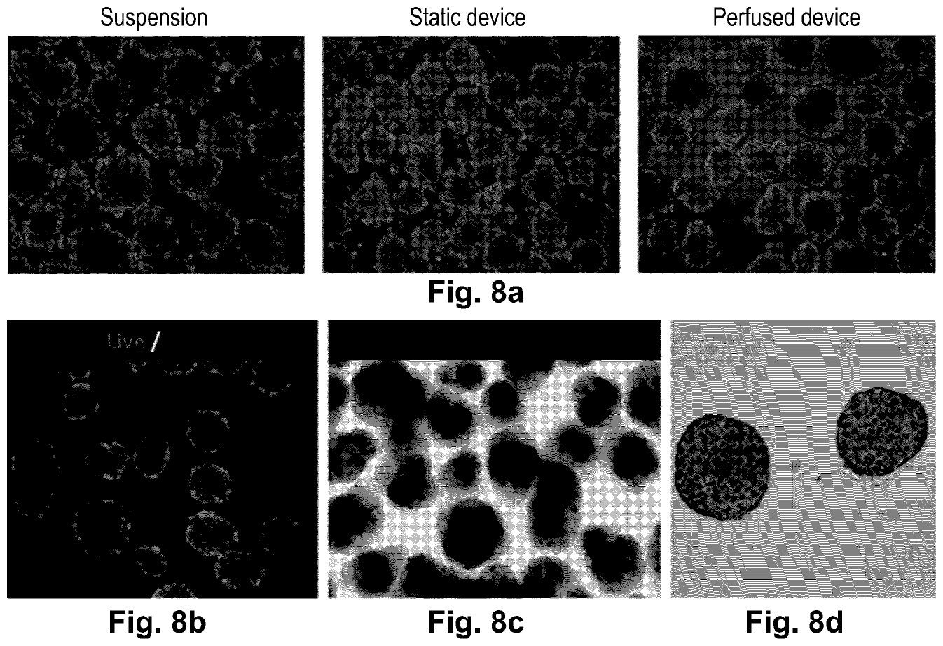

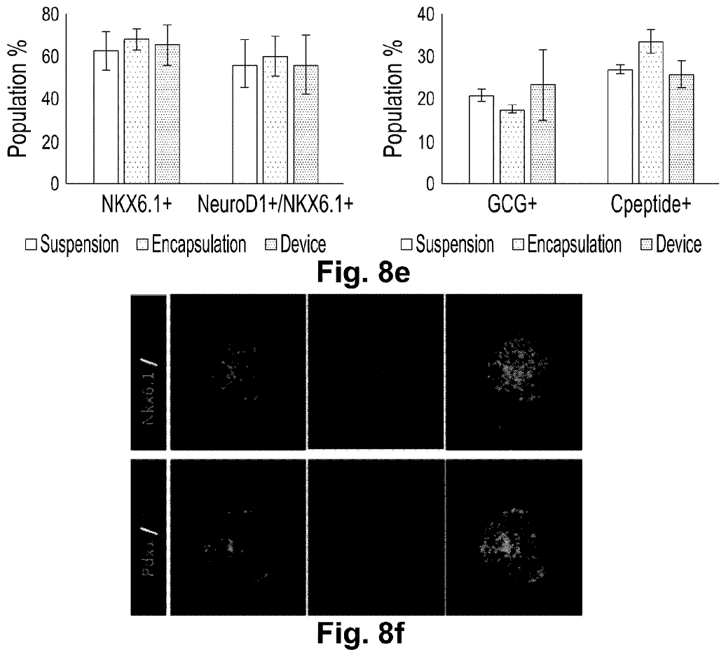

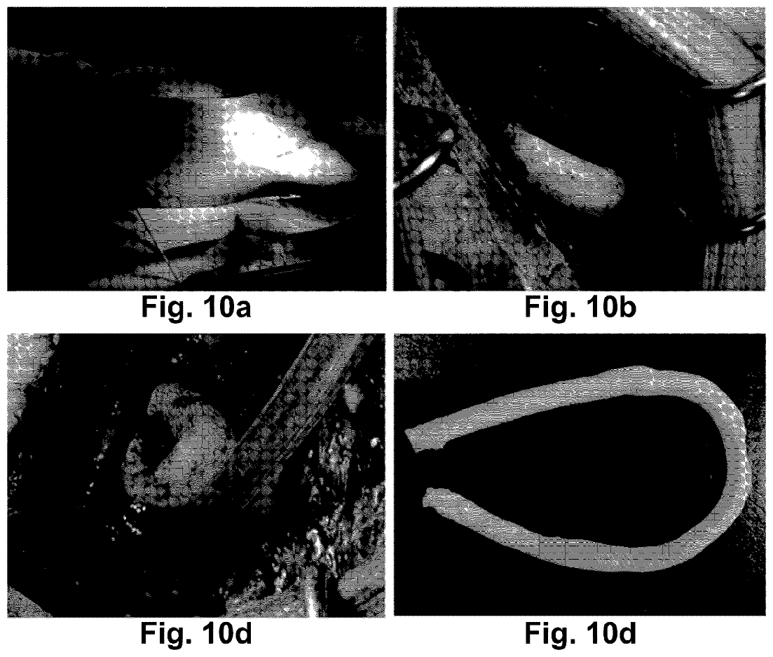

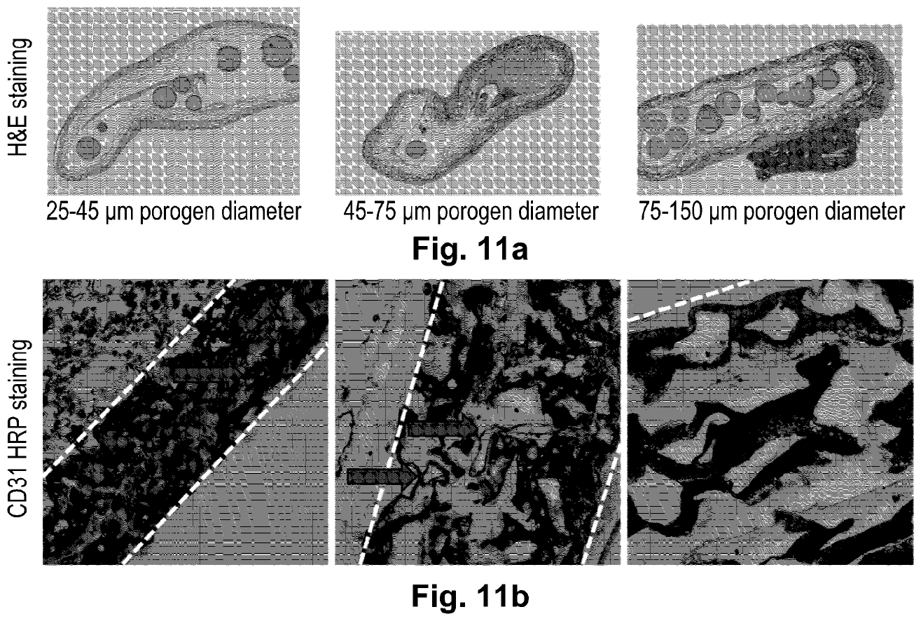

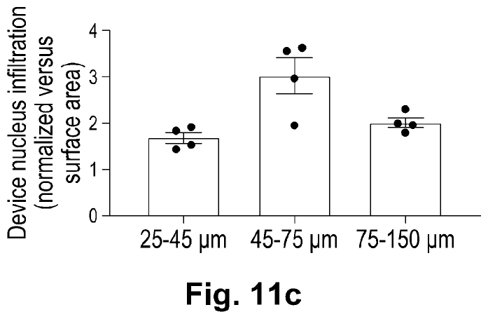

As demonstrated herein, micro- (FIG. 1) and macroencapsulation-based (FIG. 4) immune protection approaches are possible embodiments of the provided device. However, other immunoisolation strategies that are compatible with this device would be considered in other embodiments. One example is conformal coating to create nano-thin layers around the graft cells. Immunoprotection could be provided by the device material itself by further reducing porogen diameter and coating pore sizes to avoid immune cell passage through the device material. Immunoprotection may also not be needed for certain autologous grafts, immunoevasive or immunocloaked cells (e.g., by genetically modifying surface antigens), through co-transplantation of immunologically privileged cells (e.g., mesenchymal stem/stromal cells, Sertoli cells), by engineering the cell immobilization matrix to inhibit or abate immune responses, through a milder immunosuppressive regimen administered to the recipient, or through a combination thereof. As shown in FIG. 3, iterative molding and coating can be achieved using combination of 3D printed and molded sacrificial material (carbohydrate shown here) in combination with molding technologies that can also be achieved via 3D printing (PLA shown here) or molding (PDMS shown here). In addition, although stem cell derived pseudoislets survival and maturation is shown herein (FIG. 8), other cell sources can be used, either from pancreatic origin (human islets, pig islet, pancreatic organoids) or from other organs such as but not limited to liver, blood and thyroid. As shown in FIG. 10, devices can safely be implanted as arteriovenous shunts for at least one week in pigs without significant complications. Other implantation sites as shunts or interposition grafts can also be envisioned including but not restricted to the iliac or epigastric circulation.

While the present disclosure has been described in connection with specific embodiments thereof, it will be understood that it is capable of further modifications and this application is intended to cover any variations, uses, or adaptations and including such departures from the present disclosure as come within known or customary practice within the art and as may be applied to the essential features, and as follows in the scope of the appended claims.

What is claimed is:

1: A method to manufacture a cell macroencapsulation device comprising the steps of:
preparing a porous membrane by coating a sacrificial template with a non-hydrolysable polymeric solution mixed with porogen; and
encapsulating cells in the porous membrane.

2: The method of claim 1, wherein the sacrificial template is made of sugar.

3: The method of claim 1, wherein the polymeric solution consists of a polycarbonate polyurethane dissolve into a volatile organic solvent.

4: The method of claim 1, wherein the sacrificial template is coated with 1 to 10 layers of the polymeric solution mixed with porogen.

5: The method of claim 1, wherein sacrificial templates and the coatings are alternated to yield multiple compartments.

6: The method of claim 1, wherein the porous membrane is created using a molded or a 3D printed template.

7: The method of claim 6, wherein the porous membrane is made in silicon elastomer or is molded as a pouch.

8: (canceled)

9: (canceled)

10: (canceled)

11: The method of claim 1, wherein the porous membrane is further sterilized.

12: The method of claim 1, wherein the porous membrane is further dried before encapsulating the cells.

13: The method of claim 1, wherein the cells are mixed with an immobilization, immunoprotective or vasculogenic material for encapsulation into the porous membrane.

14: The method of claim 1, wherein the porogen has particle sizes ranging from 1 microns to 1000 microns in size.

15: The method of claim 14, wherein the immobilization material is an immunoprotective hydrogel.

16: (canceled)

17: (canceled)

18: (canceled)

19: (canceled)

20: The method of claim 15, wherein the hydrogel is alginate, chemically-modified alginate, agarose, chitosan, collagen, Matrigel, fibrinogen, laminins, decellularized product, a polyethyelene-glycol based gel, or blends thereof.

21: The method of claim 1, wherein the cells are encapsulated using a membrane emulsification and internal gelation.

22: The method of claim 1, wherein the cells are encapsulated using a nozzle-based encapsulation.

23: The method of claim 1, wherein the cells are encapsulated using conformal coating.

24: The method of claim 1, comprising a nanothin coating around the cells or cell aggregates.

25: (canceled)

26: A cell macroencapsulation device produced by the method of claim 1 comprising cells retained by a polymeric membrane fabricated through solvent casting the non-hydrolysable polymeric solution applied to the sacrificial template, with or without incorporation of porogens for particulate leaching.

27: (canceled)

28: The device of claim 26, wherein the membrane is a pouch or a vascular graft.

29: (canceled)

30: (canceled)

31: (canceled)

32: (canceled)

33: (canceled)

34: (canceled)

35: (canceled)

36: (canceled)

37: (canceled)

38: (canceled)

39: (canceled)

40: (canceled)

41: (canceled)

42: (canceled)

43: (canceled)

44: (canceled)

45: (canceled)

46: (canceled)

47: (canceled)

48: (canceled)

49: The device of claim 26, further comprising an immobilization material, wherein the immobilization material is loaded with angiogenic cells, such as endothelial progenitor cells, endothelial colony-forming cells, endothelial cells, mesenchymal stem/stromal cells, stromal cells, pericytes, microvessel fragments or combinations thereof.

50: (canceled)

51: (canceled)

52: (canceled)

53: (canceled)

54: (canceled)

55: (canceled)

56: (canceled)

57: (canceled)

58: (canceled)

59: (canceled)

60: (canceled)

61: (canceled)

62: (canceled)

63: (canceled)

64: (canceled)

65: (canceled)

66: (canceled)

67: (canceled)
